(19) United States
(12) Patent Application Publication
(10) Pub. No.: US 2021/0118532 A1
(43) Pub. Date: Apr. 22, 2021

(54) METHOD AND SYSTEM FOR PROVIDING BLOCKCHAIN-BASED MEDICAL INFORMATION PLATFORM, AND NON-TRANSITORY COMPUTER-READABLE RECORDING MEDIUM

(71) Applicant: MediBloc Co., Ltd., Seoul (KR)

(72) Inventors: Woo Kyun KHO, Seoul (KR); Eun Sol LEE, Seoul (KR)

(73) Assignee: MediBloc Co., Ltd., Seoul (KR)

(21) Appl. No.: 17/045,573

(22) PCT Filed: Apr. 10, 2018
(86) PCT No.: PCT/KR2018/004219

§ 371 Date: Dec. 22, 2020

(51) Int. Cl. G16H 10/60; G06F 16/27; G06F 21/60; G16H 50/70

(52) U.S. Cl. CPC G16H 10/60; G16H 50/70; G06F 21/602; G06F 16/27

(57) ABSTRACT

According to one aspect of the present invention, there is provided a method for providing a blockchain-based medical information platform, the method comprising the steps of: managing data input/output between a core layer consisting of a distributed database for storing encrypted medical information and an application layer for providing an application program utilizing the medical information, with reference to information stored on a blockchain; and providing a smart contract for managing at least one of information on tokens or points, information on user accounts, and information on a location where the medical information is stored, with reference to the information stored on the blockchain.

### FIELD OF THE INVENTION

The present invention relates to a method, system, and non-transitory computer-readable recording medium for providing a blockchain-based medical information platform.

### BACKGROUND

Conventionally, medical information is centrally managed by individual medical institutions that treat patients, and it is not allowed in principle to share the medical information outside of the medical institutions, except when the patients request their own medical records. Such a medical information system centered on individual medical institutions results in fragmentation of the medical information of the patients, which makes it difficult to utilize the medical information and deteriorates quality of medical services.

Demand for sharing of medical information has been constantly made by medical community, medical industry, patients, and the like. Although various projects for the medical information sharing have been ongoing for a long time, no clear solution has been presented. Examples thereof include the Blue Button Connector by the United States government, mobile health applications by Apple, and health applications by Samsung Electronics. Although various services other than the foregoing have been presented, the health care services so far have not fulfilled the requirements of an ideal medical information system such as security, reliability, and openness, and have consequently failed to draw active participation of medical consumers, medical providers, and healthcare related organizations or companies.

Medical information platforms using blockchain technology capable of ensuring reliability and transparency have emerged in recent years and attracted attention from many people. Multiple parties with different interests such as medical researchers, medical providers (e.g., hospitals, doctors), and general users (e.g., patients) coexist on such a platform, and the platform is not centered on any one party but based on decentralization. Thus, in order to continuously develop and maintain the platform, methods of managing and operating the platform are more important than anything else.

However, it has been difficult in the prior art to identify whether a user who handles sensitive personal information such as medical information on a decentralized medical information platform is truly reliable. Further, although active participation of users is essential for smooth operation of such a platform, conventional methods of rewarding with tokens or giving commissions performed on various platforms have been insufficient to draw active participation of the users.

In this connection, the inventor(s) present a technique for providing a blockchain-based medical information platform.

### SUMMARY OF THE INVENTION

One object of the present invention is to solve all the above-described problems in the prior art.

Another object of the invention is to create a sustainable and wholesome medical information platform by managing data input/output between a core layer consisting of a distributed database for storing encrypted medical information and an application layer for providing an application program utilizing the medical information, with reference to information stored on a blockchain, and providing a smart contract for managing at least one of information on tokens or points, information on user accounts, and information on a location where the medical information is stored, with reference to the information stored on the blockchain.

Yet another object of the invention is to draw active participation of users in a medical information platform.

Still another object of the invention is to provide criteria for directly or indirectly judging the reliability of users who handle sensitive personal information such as medical information on a medical information platform.

Still yet another object of the invention is to provide an environment in which various services for parties related to medical information, including medical consumers, may be developed using a medical information platform.

The representative configurations of the invention to achieve the above objects are described below.

According to one aspect of the invention, there is provided a method for providing a blockchain-based medical information platform, the method comprising the steps of: managing data input/output between a core layer consisting of a distributed database for storing encrypted medical information and an application layer for providing an application program utilizing the medical information, with reference to information stored on a blockchain; and providing a smart contract for managing at least one of information on tokens or points, information on user accounts, and information on a location where the medical information is stored, with reference to the information stored on the blockchain.

According to another aspect of the invention, there is provided a system for providing a blockchain-based medical information platform, the system comprising: a medical information management unit configured to manage data input/output between a core layer consisting of a distributed database for storing encrypted medical information and an application layer for providing an application program utilizing the medical information, with reference to information stored on a blockchain; and a smart contract management unit configured to provide a smart contract for managing at least one of information on tokens or points, information on user accounts, and information on a location where the medical information is stored, with reference to the information stored on the blockchain.

In addition, there are further provided other methods and systems to implement the invention, as well as non-transitory computer-readable recording media having stored thereon computer programs for executing the methods.

According to the invention, it is possible to create a sustainable and wholesome medical information platform so that a medical information system centered on medical institutions may be converted into a medical information system centered on medical consumers (i.e., patients) to enable reliable, transparent, and secure exchange of medical information, which cannot be achieved by the conventional system.

According to the invention, it is possible to create an environment in which high-quality and diverse medical information-related services may be developed on the basis of medical information accumulated on a medical information platform.

According to the invention, it is possible to draw active participation of users in a medical information platform since opportunities for predetermined rewards may be given to all users who participate in the platform.

According to the invention, it is possible to directly or indirectly judge the reliability of users who handle sensitive personal information such as medical information on the basis of points or tokens.

### BRIEF DESCRIPTION OF THE DRAWINGS

FIG. 1 schematically shows the configuration of an entire system for providing a blockchain-based medical information platform according to one embodiment of the invention.

FIGS. 2 and 3 illustratively show the internal configurations of a medical information platform according to one embodiment of the invention.

FIG. 4 illustratively shows how to calculate an exchange ratio between tokens and points according to one embodiment of the invention.

### DETAILED DESCRIPTION OF THE INVENTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

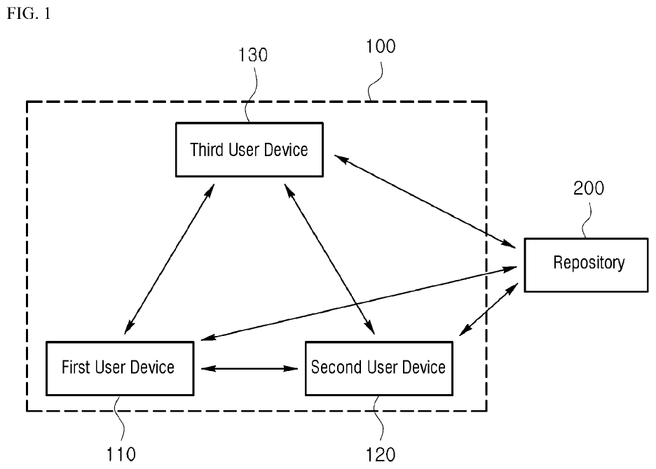

Configuration of the entire system FIG. 1 schematically shows the configuration of the entire system for providing a blockchain-based medical information platform according to one embodiment of the invention.

As shown in FIG. 1, the entire system according to one embodiment of the invention may comprise a communication network, a plurality of user devices 100, and a repository 200.

First, the communication network according to one embodiment of the invention may be configured regardless of communication modality such as wired and wireless communications, and may be constructed from a variety of communication networks such as local area networks (LANs), metropolitan area networks (MANs), and wide area networks (WANs). Preferably, the communication network described herein may be the Internet or the World Wide Web (WWW). However, the communication network is not necessarily limited thereto, and may at least partially include known wired/wireless data communication networks, known telephone networks, or known wired/wireless television communication networks.

For example, the communication network may be a wireless data communication network, at least a part of which may be implemented with a conventional communication scheme such as radio frequency (RF) communication, WiFi communication, cellular communication (e.g., Long Term Evolution (LTE) communication), Bluetooth communication (more specifically, Bluetooth Low Energy (BLE) communication), infrared communication, and ultrasonic communication.

Next, the plurality of devices 100 according to one embodiment of the invention are digital equipment that may function to communicate with each other via the communication network, and any type of digital equipment having a memory means and a microprocessor for computing capabilities, such as a computer, a laptop, a smart phone, and a tablet PC, may be adopted as the plurality of devices 100 according to the invention.

Meanwhile, the plurality of devices 100 according to one embodiment of the invention may include an operating system (to be described below) for providing a blockchain-based medical information platform according to the invention, in the form of a program module such as an application or a widget. Further, the program module may be downloaded from an external application distribution server (not shown), an external system (not shown), or the like.

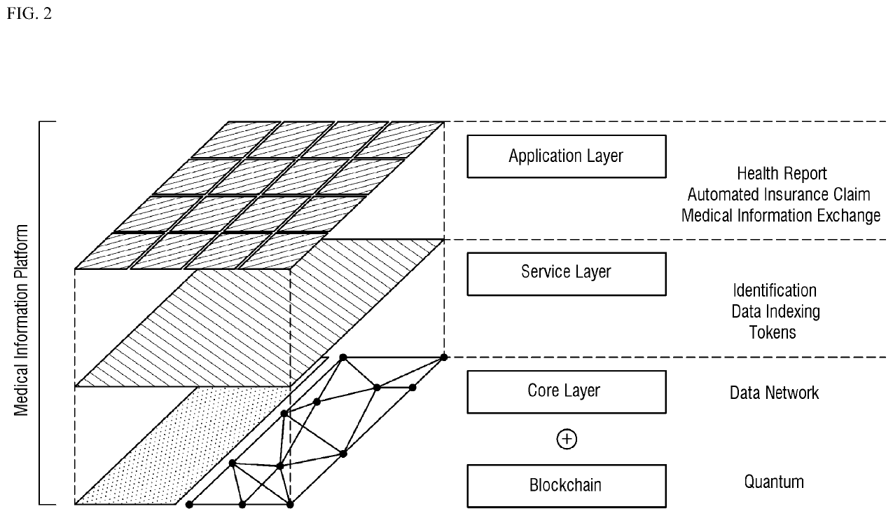

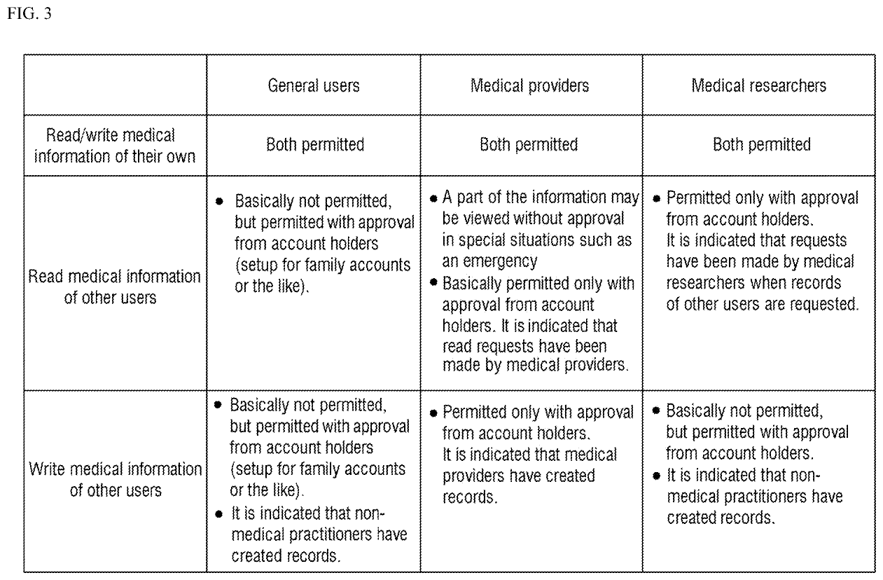

FIGS. 2 and 3 illustratively show the internal configurations of a medical information platform according to one embodiment of the invention.

First, referring to FIG. 2, the medical information platform according to one embodiment of the invention may comprise a core layer, a service layer, and an application layer, and may operate based on a Quantum blockchain among known blockchains.

The core layer according to one embodiment of the invention may function as a distributed database that may safely protect medical information of users using encryption technology (e.g., by encrypting the medical information through private keys that may be decrypted only by the users corresponding to the medical information). Further, a separate storage space may be required in order to store or manage the medical information of the users since the amount of information that may be stored in the blockchain is limited, and the core layer may function to manage the repository 200 (i.e., distributed database) to this end.

Meanwhile, according to one embodiment of the invention, the medical information of the users provided to the core layer through the application layer may be encrypted by the application layer.

Further, the service layer according to one embodiment of the invention may enable the application layer and the core layer to be connected to each other, and may function to manage user information (e.g., user accounts for identifying the users), index the medical information, and so on. Furthermore, the service layer according to one embodiment of the invention may be connected to the core layer through the medical information stored on the blockchain (e.g., the medical information may be stored in the repository 200 when it is large and a corresponding hash value may be stored on the blockchain, wherein the service layer may find from the core layer a location where the medical information corresponding to the hash value is stored), and may function to input/output the medical information to/from the core layer.

Meanwhile, the service layer according to one embodiment of the invention may further provide a smart contract based on an Ethereum virtual machine (EVM). (For example, the smart contract may include information on a user's account, information on a link related to a location where the medical information is stored, and the like). In other words, according to one embodiment of the invention, the service layer may comprise a part providing the smart contract and a part connecting the application layer and the core layer.

Further, the application layer according to one embodiment of the invention may include various types of application programs that may run on the user devices 100 to manage the medical information platform.

Referring to FIG. 3, permission for encrypted medical information (e.g., permission to read (or view) or write (or create) the medical information) may be set on the medical information platform, on the basis of types of users who use the platform.

For example, according to one embodiment of the invention, the types of the users may include general users, medical providers, and medical researchers. According to one embodiment of the invention, the general users, the medical providers, and the medical researchers may all be granted permission to read or write the medical information of their own, and permission to read or write the medical information of other users may be basically set such that the medical information cannot be accessed without separate approval from the other users. However, according to one embodiment of the invention, the medical providers may be temporarily granted permission to read a part of the medical information of other users, in predetermined situations such as an emergency. Meanwhile, the medical providers and the medical researchers may be granted permission to request other users to grant permission to read the medical information of the other users.

Meanwhile, according to one embodiment of the invention, at least one of a conventional centralized method to get direct authentication from a trusted organization and a peer-to-peer (P2P) method to get authentication from an authenticated user may be used to classify the types of the users on the medical information platform.

According to one embodiment of the invention, the P2P authentication method may be determined by voting of a plurality of authenticated users (in this case, voting results of individual users may not be revealed through an anonymous indorsement protocol (AIP) using a zero-knowledge proof method defined by Indorse, for example). Further, according to one embodiment of the invention, in order to increase the reliability of the P2P authentication method, predetermined points may be paid to an authenticated user participating in an authentication process of another user, in the form of a deposit (or collateral). The points paid in the form of the deposit may be confiscated from (or predetermined points or tokens may be imposed as a fine to) any user who is judged to be malicious or undermine trust of the authentication process, while predetermined points may be given as a reward to any user who is judged to perform the authentication process in good faith or in a normal manner.

Meanwhile, according to one embodiment of the invention, a user may search for medical information of other users through search on the medical information platform. Of course, all users have the right to refuse the disclosure or search of their medical information, and thus only medical information of users who have allowed the search may be searched.

Specifically, according to one embodiment of the invention, an opportunity to participate in medical information trade may be given to a user who does not want his/her medical information to be directly searched, but is willing to sell or donate the medical information when predetermined conditions are met. Further, according to one embodiment of the invention, a user who wants to obtain medical information of other users may perform a direct search, but may also notify other users on the medical information platform that the user is seeking specific medical information, with his/her desired conditions and compensation requirements being specified. In addition, according to one embodiment of the invention, a user may determine whether his/her medical information meets the specified conditions personally (or with the help of the medical information platform), and then notify other users on the medical information platform that the user is willing to trade the medical information, through a push notification function or the like. Meanwhile, according to one embodiment of the invention, at least a part of the above medical information trade processes may be performed in a background without necessarily requiring active participation of the corresponding user.

Meanwhile, according to one embodiment of the invention, predetermined security technology (e.g., Software Guard Extensions of Intel) may be applied in order to minimize the exposure of medical information to users and prevent even an administrator of the operating system from recognizing medical information of users.

Meanwhile, the medical information platform according to one embodiment of the invention may refer to a decentralized platform capable of sharing encrypted medical information among a plurality of users on the basis of blockchain technology.

Meanwhile, according to one embodiment of the invention, the medical information platform may be implemented based on a variety of conventional blockchains such as Bitcoin, Ethereum, and Quantum.

In addition, according to one embodiment of the invention, the blockchain based on which the medical information platform is implemented may encompass various types of blockchains such as a private blockchain, a public blockchain, or a combination of private and public blockchains.

Further, the medical information platform according to one embodiment of the invention may issue tokens for platform operation or the like. For example, according to one embodiment of the invention, the tokens may be generated on the basis of the conventional Ethereum token standard (i.e., ERC-20) or Quantum token standard (i.e., QRC, a Quantum's counterpart of ERC-20).

Furthermore, the medical information platform according to one embodiment of the invention may include a token pool for integrated management of tokens.

For example, according to one embodiment of the invention, the token pool may include (1) newly issued tokens (for example, after an initial coin offering (ICO) on the medical information platform, the inflation rate applied to the newly issued tokens may be set to be 5% in the first year and to decrease by 30% from the following year), (2) tokens that users pay for using services provided by the medical information platform, and (3) tokens that users exchange for points through the operating system according to the invention.

Meanwhile, according to one embodiment of the invention, the tokens included in the token pool may be used as tokens for which users exchange points through the operating system according to the invention, and may be used to pay for operation, maintenance, and the like of the medical information platform.

Further, the medical information platform according to one embodiment of the invention may further issue points that may be exchanged with tokens, and these points may be used as indicators of extents of users' contributions to the platform. That is, according to one embodiment of the invention, on the medical information platform, predetermined points may be given as a reward to a user who is determined to perform a certain activity meaningful to the platform, on the basis of the extent of the user's contribution.

According to one embodiment of the invention, the above points may be given at every predetermined period (e.g., one month) and may be expired after a predetermined period (e.g., 24 months) from when they are given. Through the foregoing, unnecessary inflation due to unused points may be prevented and the medical information platform may remain healthy.

Further, according to one embodiment of the invention, the points given as above may be set to be exchanged for the tokens after a predetermined reserve period (e.g., three months).

Meanwhile, according to one embodiment of the invention, on the medical information platform, tokens may be set to be traded with other users while points may be set not to be traded with other users. That is, according to one embodiment of the invention, points may not be acquired through trade with other users unlike tokens, and may be set to be acquired by contributing to the platform or paying the platform a large amount of tokens to be exchanged for points. Accordingly, in trading sensitive medical information with other parties including medical institutions or healthcare companies, points may at least serve as an objective indicator that may be used to judge the reliability of the trading parties.

Next, the operating system according to one embodiment of the invention may function to dynamically calculate an exchange ratio between tokens and points, with reference to at least one of an amount of points that a user intends to exchange for tokens on a medical information platform, an amount of tokens that the user intends to exchange for points on the medical information platform, an amount of tokens present in a token pool managed by the medical information platform, and an amount of points previously exchanged for tokens on the medical information platform, and to provide the user with the tokens or points exchanged with reference to the calculated exchange ratio.

The configurations and functions of the operating system according to the invention will be discussed in more detail below. Meanwhile, although the operating system has been described as above, the above description is illustrative and it will be apparent to those skilled in the art that at least a part of the functions or components required for the operating system may be implemented or included in the plurality of devices 100 (e.g., a first user device 110, a second user device 120, and a third user device 130) or an external system (not shown), as necessary. For example, according to one embodiment of the invention, a user may exchange tokens and points according to the invention by installing an application including at least a part of the functions of the operating system (or medical information platform) according to the invention on his/her device, or connecting to a website providing at least at least a part of the functions of the operating system (or medical information platform) according to the invention using his/her device.

Next, the repository 200 according to one embodiment of the invention may be connected with the medical information platform via the communication network, and may function to encrypt and store medical information. For example, according to one embodiment of the invention, the repository 200 may encrypt and store medical information through a private key that may be decrypted only by a user corresponding to the medical information, and a corresponding hash value may be stored on the blockchain implemented on the service layer.

Meanwhile, the repository 200 according to one embodiment of the invention may be a decentralized repository configured on the basis of interplanetary file system (IPFS).

Configuration of the Operating System

Hereinafter, the functions of the respective components of the operating system crucial for implementing the invention will be discussed.

The operating system according to one embodiment of the invention may comprise a medical information management unit, a smart contract management unit, an exchange ratio management unit, and a token/point provision unit. According to one embodiment of the invention, at least some of the medical information management unit, the smart contract management unit, the exchange ratio management unit, and the token/point provision unit may be program modules to communicate with an external system. The program modules may be included in the operating system in the form of operating systems, application program modules, and other program modules, while they may be physically stored in a variety of commonly known storage devices. Further, the program modules may also be stored in a remote storage device that may communicate with the operating system. Meanwhile, such program modules may include, but not limited to, routines, subroutines, programs, objects, components, data structures, and the like for performing specific tasks or executing specific abstract data types as will be described below in accordance with the invention.

First, the medical information management unit according to one embodiment of the invention may function to manage data input/output between a core layer consisting of a distributed database for storing encrypted medical information and an application layer for providing an application program utilizing the medical information, with reference to information stored on a blockchain.

Next, the smart contract management unit according to one embodiment of the invention may function to provide a smart contract for managing at least one of information on tokens or points, information on user accounts, and information on a location where the medical information is stored, with reference to the information stored on the blockchain.

Next, the exchange ratio management unit according to one embodiment of the invention may function to dynamically calculate an exchange ratio between tokens and points, with reference to at least one of an amount of points that a user intends to exchange for tokens on a medical information platform, an amount of tokens that the user intends to exchange for points on the medical information platform, an amount of tokens present in a token pool managed by the medical information platform, and an amount of points previously exchanged for tokens on the medical information platform.

Specifically, when a user intends to exchange at least a part of his/her points for tokens on the medical information platform, the exchange ratio management unit according to one embodiment of the invention may dynamically calculate a token exchange ratio of the points to be exchanged, on the basis of an amount of the points to be exchanged and the amount of tokens present in the token pool.

For example, the exchange ratio management unit according to one embodiment of the invention may calculate a ratio at which points are exchanged for tokens, on the basis of a ratio between a predetermined percentage of the amount of tokens held in the token pool and the amount of points that the user intends to exchange for tokens.

More specifically, the exchange ratio management unit according to one embodiment of the invention may calculate the ratio at which points are exchanged for tokens as a value obtained by dividing 30% of the amount of tokens held in the token pool by the amount of points that the user intends to exchange for tokens.

Further, when the user intends to exchange at least a part of his/her tokens for points, the exchange ratio management unit according to one embodiment of the invention may dynamically calculate a point exchange ratio of the tokens to be exchanged, on the basis of an amount of points previously exchanged for tokens by the user on the medical information platform.

For example, the exchange ratio management unit according to one embodiment of the invention may calculate a ratio at which tokens are exchanged for points, by calculating the amount of points previously exchanged for tokens by the user on the medical information platform, using at least one of a linear function and a logarithmic function.

More specifically, the exchange ratio management unit according to one embodiment of the invention may calculate the ratio at which tokens are exchanged for points, by calculating the amount of points previously exchanged for tokens on the medical information platform, using a linear function when the amount is not greater than a predetermined level, and using a logarithmic function when the amount is greater than the predetermined level.

Next, the token/point provision unit according to one embodiment of the invention may function to provide the user with the tokens or points exchanged with reference to the calculated exchange ratio between tokens and points.

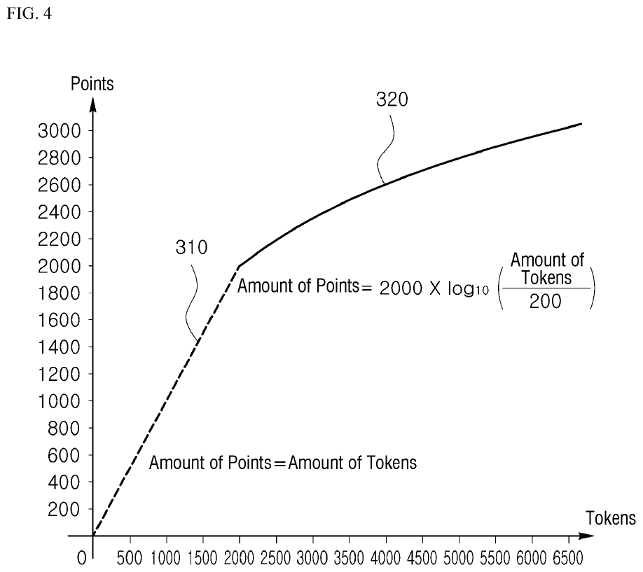

FIG. 4 illustratively shows how to calculate an exchange ratio between tokens and points according to one embodiment of the invention.

Referring to FIG. 4, the operating system according to one embodiment of the invention may calculate a point exchange ratio of tokens as 1:1 until an amount of points previously exchanged for tokens by a user reaches 2,000 (310), and may calculate the point exchange ratio of tokens such that an amount of exchanged points is 2,000×log₁₀ (amount of tokens/200) when the amount of points previously exchanged for tokens by the user is greater than 2,000.

That is, according to one embodiment of the invention, users may acquire basic points by exchanging the same amount of tokens, but should use a large amount of tokens in order to acquire more points, so that the users are induced to acquire points through certain meaningful activities (i.e., contributions) on the platform.

Meanwhile, the formula for calculating the ratio at which tokens are exchanged for points according to the invention is not necessarily limited to the foregoing, and may be changed to various formulas as long as the objects of the invention may be achieved.

### VARIOUS EMBODIMENTS

Hereinafter, various services that may be implemented using the medical information platform according to the invention will be discussed.

1. Personal Health Report

According to one embodiment of the invention, a personal health report service may be provided which assists medical consumers to integrate and manage medical records scattered across multiple medical providers and vast medical-related information collected through various wearable devices, using the medical information platform according to the invention.

Specifically, according to one embodiment of the invention, the medical information integrated as above may be managed and used as records for health of the medical consumers, and may be utilized when the medical consumers receive treatment or personalized healthcare services including artificial intelligence.

Further, according to one embodiment of the invention, the medical consumers may easily check information on when and for what purposes they visited hospitals, what kind of treatment they received, and what their conditions are, as well as information on what kind of medicine they are taking, what ingredients the medicine consists of, and what effects (or side effects) the medicine has. In addition, according to one embodiment of the invention, the medical consumers may also obtain information on how their health conditions are changed and what their current health conditions are, through comparison with past medical records.

2. Automatic Insurance Claim

According to one embodiment of the invention, an automatic insurance claim service may be provided which assists automatic insurance claim and examination using medical records collected through the medical information platform according to the invention and a smart contract provided by the medical information platform according to the invention. According to the automatic insurance claim service according to the invention, an insurant no longer needs to call an insurance company or visit an insurance agent to ask about what diseases, tests, and treatments are covered by the insurance of the insurant, and to submit his/her medical records to the insurance company one by one.

Specifically, according to one embodiment of the invention, the insurance claim may be automatically made according to the insurance of the insurant, using the medical records and smart contract provided after the insurant receives treatment, and the medical records are compared with the insurance contract to determine whether the insurance claim may be made for any coverage items of the insurance contract, so that the insurance money may be paid to the insurant and a personalized insurance product may be designed and recommended on the basis of the integrated medical records.

3. P2P Healthcare Data Market

According to one embodiment of the invention, a P2P medical data market service may be provided which assists medical consumers to be directly connected to medical researchers, medical institutions, companies, and the like wanting their medical data (i.e., medical information) through the medical information platform according to the invention.

Some large medical institutions or companies have monopolized the distribution of medical information and financial benefits therefrom so far. However, according to the P2P medical data market service according to the invention, the benefits that may be obtained by using medical data may be returned to the medical consumers who own the medical data. Further, according to the P2P medical data market service according to the invention, high-quality medical data may be generated and a market may be created in which medical data distribution is centered on patients who own medical data, so that medical data accessibility may be enhanced.

4. Artificial Intelligence

According to one embodiment of the invention, an artificial intelligence service may be provided which is significantly advanced on the basis of a large amount of high-quality medical information acquired through the medical information platform according to the invention.

Specifically, according to the artificial intelligence service according to the invention, a medical consumer may receive a personalized artificial intelligence service by providing his/her medical information to an artificial intelligence service such as a medical chatbot. For example, the medical consumer may receive a service for recommending a necessary medical practitioner, a service for estimating a diagnosis, a service for recommending a treatment, and a service for predicting a prognosis.

5. Clinical Trials

According to one embodiment of the invention, a service helpful for medical research institutions, pharmaceutical companies, and the like to conduct clinical trials may be provided by selecting and recommending subjects suitable for the clinical trials on the basis of medical information acquired through the medical information platform according to the invention.

For example, when a prospective study is conducted, a subject may grant permission to read medical information related to the study to researchers, research supervisors, institutions, and the like through a smart contract. This makes it possible to more objectively verify the study while the study is conducted.

As another example, when a retrospective study is conducted, a researcher may find a subject that meets his/her desired conditions on the medical information platform, and obtain necessary medical information from the subject to conduct the study.

6. Telemedicine

According to one embodiment of the invention, a telemedicine service may be provided which is capable of providing necessary medical services to medical consumers over physical/temporal barriers, using abundant medical resources connected through the medical information platform according to the invention.

According to the telemedicine service according to the invention, a medical consumer may be connected to a desired medical practitioner and receive a necessary service from the medical practitioner, all over the world around the clock.

7. Social Networking Service (SNS)

According to one embodiment of the invention, a social networking service may be provided which assists medical consumers or patients with similar diseases (e.g., rare diseases) to share information or form ties with each other, by creating a community consisting of the medical consumers and enabling communication between the medical consumers, using medical information acquired through the medical information platform according to the invention.

According to the social networking service according to the invention, the medical consumers may receive great emotional help through communication with each other, and medical providers, researchers, and the like may naturally participate in the community consisting of the medical consumers.

The embodiments according to the invention as described above may be implemented in the form of program instructions that can be executed by various computer components, and may be stored on a computer-readable recording medium. The computer-readable recording medium may include program instructions, data files, data structures and the like, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be configured to operate as one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for providing a blockchain-based medical information platform, the method comprising the steps of:
managing data input/output between a core layer consisting of a distributed database for storing encrypted medical information and an application layer for providing an application program utilizing the medical information, with reference to information stored on a blockchain; and
providing a smart contract for managing at least one of information on tokens or points, information on user accounts, and information on a location where the medical information is stored, with reference to the information stored on the blockchain.

2. The method of claim 1, wherein a hash value of the medical information is stored on the blockchain.

3. The method of claim 1, wherein the medical information is created or read by at least one of general users, medical providers, and medical researchers.

4. The method of claim 1, wherein a predetermined reward is provided to a user on the basis of an extent of a contribution to the medical information platform.

5. A non-transitory computer-readable recording medium having stored thereon a computer program for executing the method of claim 1.

6. A system for providing a blockchain-based medical information platform, the system comprising:
a medical information management unit configured to manage data input/output between a core layer consisting of a distributed database for storing encrypted medical information and an application layer for providing an application program utilizing the medical information, with reference to information stored on a blockchain; and
a smart contract management unit configured to provide a smart contract for managing at least one of information on tokens or points, information on user accounts, and information on a location where the medical information is stored, with reference to the information stored on the blockchain.
